(19) United States
(12) Patent Application Publication
(10) Pub. No.: US 2025/0082706 A1
(43) Pub. Date: Mar. 13, 2025

(54) FERMENTED GINSENG RADIX ET RHIZOMA EXTRACT, PRODUCT FOR ALLEVIATING NON-ALCOHOLIC FATTY LIVER DISEASE, METHOD FOR PROMOTING INCREASE OF CONTENT OF AGLYCONE FLAVONOIDS AND/OR SHORT-CHAIN FATTY ACIDS IN GINSENG RADIX ET RHIZOMA EXTRACT, AND METHOD FOR PROMOTING EFFICACY OF GINSENG RADIX ET RHIZOMA EXTRACT IN ALLEVIATING NON-ALCOHOLIC FATTY LIVER DISEASE

(71) Applicant: INFINITUS (CHINA) COMPANY LTD., Guangdong (CN)

(72) Inventors: Cencen LIU, Guangdong (CN); Shumao CUI, Guangdong (CN); Hongwei ZHAO, Guangdong (CN); Jian TANG, Guangdong (CN); Fei LIU, Guangdong (CN)

(73) Assignee: INFINITUS (CHINA) COMPANY LTD., Guangdong (CN)

(21) Appl. No.: 18/746,083

(22) Filed: Jun. 18, 2024

(30) Foreign Application Priority Data
Sep. 13, 2023 (CN) 202311184003.0

(51) Int. Cl. A61K 36/258; A61P 1/16; A61P 3/06

(52) U.S. Cl. CPC A61K 36/258; A61P 1/16; A61P 3/06; A61K 2236/19; A61K 2236/53

(57) ABSTRACT

The present invention discloses use of Bifidobacterium animalis subsp.lactis in promoting efficacy exertion of a Ginseng Radix Et Rhizoma extract in alleviating liver injuries. According to the present invention, Bifidobacterium animalis subsp.lactis CCFM1274 may grow in a fermentation base rich in a Ginseng Radix Et Rhizoma extract, and a fermentation broth of the Ginseng Radix Et Rhizoma extract fermented with this strain can increase the content of active substances such as aglycone flavonoids and short-chain fatty acids, promote the efficacy exertion of the Ginseng Radix Et Rhizoma extract, including improving the ability of the Ginseng Radix Et Rhizoma extract to alleviate a non-alcoholic liver injury, and reduce the fat accumulation in liver cells. This strain and the Ginseng Radix Et Rhizoma extract may be used to prepare a product for promoting the efficacy of alleviating the non-alcoholic liver injury.

### CROSS-REFERENCE TO RELATED APPLICATION

This application claims the priority benefit of China application serial no. 202311184003.0, filed on Sep. 13, 2023. The entirety of the above-mentioned patent application is hereby incorporated by reference herein and made a part of this specification.

### TECHNICAL FIELD

The present invention belongs to the technical field of microorganisms. More particularly, the present invention relates to use of Bifidobacterium animalis subsp.lactis CCFM1274 in promoting the efficacy exertion of a Ginseng Radix Et Rhizoma extract, including use in promoting the efficacy exertion of the Ginseng Radix Et Rhizoma extract in alleviating a non-alcoholic metabolic liver injury.

### BACKGROUND

With the improvement of people's living conditions, unhealthy eating habits and sedentary lifestyles lead to a gradual increase in the number of patients with a non-alcoholic fatty liver disease (NAFLD). NAFLD is a chronic metabolic disease with clinical symptoms of excess lipid accumulation in the liver. The spectra of this disease include: non-alcoholic simple fatty liver, non-alcoholic fatty liver hepatitis, liver cirrhosis and liver cancer. A prevalence rate of this disease in Western countries accounts for about 20% to 30% of that of the general population. In recent years, NAFLD has also been on the rise in China and has greatly increased the chances of NAFLD patients suffering diabetes, atherosclerosis and other cardiovascular diseases, as well as liver injuries, which in severe cases can develop into liver cirrhosis, liver cancer and liver failure.

Currently, lipid-lowering drugs such as fibrates, statins and selective TC (total cholesterol) reuptake inhibitors are widely used in the clinic, which can effectively regulate the abnormalities of lipid metabolism, promote the conversion of TG, hydrolyze TC, and reduce the content of TG (triglyceride) and TC. All of them have good clinical efficacy, but a long-term taking may lead to withdrawal rebound, liver and kidney function impairments and other adverse reactions. In order to expand therapeutic schemes for NAFLD, research trends in recent years have focused on Chinese herbal extracts and active ingredients that can inhibit lipid accumulation and inflammations in liver cells.

Ginseng Radix Et Rhizoma has an extremely special status in the medical history in China, known as the “king of all herbs”, and is regarded as a landmark herb in traditional Chinese medicines. Ginseng Radix Et Rhizoma has been used in a large number of medicines, health products and cosmetics. Some studies have shown that a Ginseng Radix Et Rhizoma extract can exert certain efficacy in alleviating NAFLD, and possesses extensive value to be developed into products related to the alleviation of NAFLD.

However, it is difficult to obtain immediate results with direct use of the Ginseng Radix Et Rhizoma extract, resulting in poor efficacy exertion when the Ginseng Radix Et Rhizoma extract is eaten. Therefore, how to promote the efficacy exertion of the Ginseng Radix Et Rhizoma extract is the key to influence the development of the Ginseng Radix Et Rhizoma extract as a product related to the alleviation of NAFLD.

### SUMMARY

The technical problem to be solved by the present invention is to overcome the defects and deficiencies of the existing technology, and to provide use of Bifidobacterium animalis subsp.lactis CCFM1274 in promoting the efficacy exertion of a Ginseng Radix Et Rhizoma extract in alleviating a non-alcoholic metabolic liver injury.

An object of the present invention is to provide use of Bifidobacterium animalis subsp.lactis CCFM1274 in the preparation of a product for promoting the efficacy of a Ginseng Radix Et Rhizoma extract in alleviating a non-alcoholic fatty liver disease.

Another object of the present invention is to provide a fermented Ginseng Radix Et Rhizoma extract for promoting the efficacy of alleviating a non-alcoholic fatty liver disease.

Yet another object of the present invention is to provide a product for alleviating a non-alcoholic fatty liver disease.

Yet another object of the present invention is to provide a method for promoting an increase in the content of aglycone flavonoids and/or short-chain fatty acids in a Ginseng Radix Et Rhizoma extract.

The above objects of the present invention are achieved by the following technical solutions.

After Bifidobacterium animalis subsp.lactis CCFM1274 is cultured on a modified MRS medium for 48 h, colonies are round, convex or lenticular, slightly whitish, and opaque, with a smooth to mucus-like soft surface. This bacterium was preserved in the Guangdong Microbial Collection Center on Sep. 15, 2022, with a preservation number of GDMCC No. 62798. Fermentation of the Ginseng Radix Et Rhizoma extract using this bacterium can significantly enhance aglycone flavonoids and short-chain fatty acids (acetic acid and butyric acid), and can promote the efficacy exertion of the Ginseng Radix Et Rhizoma extract. Specifically, (1) composite fermentation broth of the Bifidobacterium animalis subsp.lactis CCFM1274 and the Ginseng Radix Et Rhizoma extract can improve the cell proliferation capacity in a HepG2 non-alcoholic cell injury and reduce fat accumulation in liver cells; (2) pretreatment of liver cells with the composite fermentation broth of the Ginseng Radix Et Rhizoma extract and this strain can improve their ability to inhibit lipid accumulation in liver cells; and (3) the Bifidobacterium animalis subsp.lactis CCFM1274 can significantly improve the ability of the Ginseng Radix Et Rhizoma extract to inhibit lipid accumulation in the liver cells. Hence:
the present invention provides use of Bifidobacterium animalis subsp.lactis CCFM1274 in the preparation of a product for promoting the efficacy of a Ginseng Radix Et Rhizoma extract for a non-alcoholic fatty liver disease.

The present invention further provides use of a combination of Bifidobacterium animalis subsp.lactis CCFM1274 and a Ginseng Radix Et Rhizoma extract in the preparation of a product for alleviating a non-alcoholic fatty liver disease.

The present invention further provides a fermented Ginseng Radix Et Rhizoma extract for promoting the efficacy of alleviating a non-alcoholic fatty liver disease, which is obtained by fermenting a Ginseng Radix Et Rhizoma extract with Bifidobacterium animalis subsp.lactis CCFM1274.

Optionally, a fermentation method includes the following steps:
step 1, activating the Bifidobacterium animalis subsp.lactis CCFM1274 to obtain a bacterial solution;
step 2, inoculating the bacterial solution of step 1 into a fermentation base containing a Ginseng Radix Et Rhizoma extract for fermentation; and
step 3, centrifuging after the fermentation is completed, and taking a fermentation supernatant.

In an embodiment of the present invention, in step 1, the Bifidobacterium animalis subsp.lactis CCFM1274 is scribed on an MRS solid medium, a flat plate is cultured inversely (preferably cultured inversely at 37° C. for 48 h), and then a single colony is picked, and inoculated and cultured (preferably cultured at 37° C. for 48 h) into an MRS liquid medium (preferably 5 mL) to obtain a bacterial solution.

In an embodiment of the present invention, in step 2, the Ginseng Radix Et Rhizoma extract in the fermentation base containing the Ginseng Radix Et Rhizoma extract has a concentration of 2-10 mg/mL (preferably 5 mg/mL).

In an embodiment of the present invention, in step 2, the bacterial solution has an inoculation amount of 1-8% (v/v).

In an embodiment of the present invention, the fermentation in step 2 is performed at a constant temperature of 30-40° C. for 24-72 h (preferably, at a constant temperature of 37° C. for 48 h).

In an embodiment of the present invention, in step 3, the centrifuging is performed in the following condition: the centrifuging is performed at 6000-10000 r/min for 10-20 min (preferably, at 8000 r/min for 15 min).

The present invention further provides use of the fermented Ginseng Radix Et Rhizoma extract in the preparation of a product for alleviating a non-alcoholic fatty liver disease.

In an embodiment of the present invention, the product is food, a drug or a health product.

In addition, the liver injuries include a non-alcoholic metabolic liver injury, and the fatty liver includes non-alcoholic fatty liver.

In the above use, the product includes at least one of the following functions:

(1) improving the proliferation activity of liver cells;

(2) reducing the number of red lipid droplets in the liver cells;

(3) increasing the content of aspartate aminotransferase (AST) and alanine aminotransferase (ALT) in the liver cells;

(4) increasing a TG clearance rate; and

(5) inhibiting the lipid accumulation in the liver cells.

The present invention further provides use of Bifzdobacterium animalis subsp.lactis CCFM1274 in promoting an increase in the content of aglycone flavonoids and/or short-chain fatty acids (acetic acid and butyric acid) in a Ginseng Radix Et Rhizoma extract, and use thereof in the preparation of a Ginseng Radix Et Rhizoma extract product with increased content of aglycone flavonoids and/or short-chain fatty acids (acetic acid and butyric acid).

The present invention further provides a method for promoting an increase in the content of aglycone flavonoids and/or short-chain fatty acids (acetic acid and butyric acid) in a Ginseng Radix Et Rhizoma extract, in which Bifidobacterium animalis subsp.lactis CCFM1274 is added to a system containing the Ginseng Radix Et Rhizoma extract.

The present invention further provides a product for alleviating a non-alcoholic fatty liver disease, which contains Bifidobacterium animalis subsp.lactis CCFM1274 and a Ginseng Radix Et Rhizoma extract.

A product for alleviating a non-alcoholic fatty liver disease contains the fermented Ginseng Radix Et Rhizoma extract.

In an embodiment of the present invention, in the product, the Bifidobacterium animalis subsp.lactis CCFM1274 added is at least 1×10⁹ CFU/ml.

In an embodiment of the present invention, the drug contains Bifidobacterium animalis subsp.lactis CCFM1274, a drug carrier and/or a pharmaceutical excipient.

In an embodiment of the present invention, the health product contains Bifidobacterium animalis subsp.lactis CCFM1274, a carrier and/or an excipient.

Based on the above content, the present invention further provides a method for promoting the efficacy exertion of the Ginseng Radix Et Rhizoma extract. In the method, Bifidobacterium animalis subsp.lactis CCFM1274 is added to a system containing the Ginseng Radix Et Rhizoma extract. The method is not for a disease diagnosis and treatment purpose. The method specifically includes: adding the Bifidobacterium animalis subsp.lactis CCFM1274 to the system containing the Ginseng Radix Et Rhizoma extract, followed by fermentation. The fermentation method is as described above.

The present invention has the following beneficial effects.

According to the present invention, the Bifidobacterium animalis subsp.lactis CCFM1274 may grow in the fermentation base rich in the Ginseng Radix Et Rhizoma extract, and the fermentation broth of the Ginseng Radix Et Rhizoma extract fermented with this strain can increase the content of active substances such as aglycone flavonoids and short-chain fatty acids (acetic acid and butyric acid), promote the efficacy exertion of the Ginseng Radix Et Rhizoma extract, including improving the ability of the Ginseng Radix Et Rhizoma extract to alleviate the non-alcoholic liver injury, and reduce the fat accumulation in liver cells.

The Bifidobacterium animalis subsp.lactis CCFM1274 and the Ginseng Radix Et Rhizoma extract can be used to produce the product for promoting the efficacy of alleviating the non-alcoholic liver injury. A total ginsenoside extract can be used as a raw material in the production process to promote the efficacy exertion of the Ginseng Radix Et Rhizoma extract. This process has the advantages of safety, high efficiency, low cost, mild reaction, and the like. This process is suitable for large-scale industrial production.

In addition, Bifidobacterium animalis is a common intestinal microorganism, which plays an important role in maintaining intestinal balance, inhibiting the growth and reproduction of harmful microorganisms in the intestines, resisting pathogen infection, enhancing the organism immunity, etc. Previous studies have shown that the Bifidobacterium animalis has many health benefits for human bodies. The Bifidobacterium animalis is also one of the most widely used Bifidobacterium species in the food industry today, especially in the dairy industry, such as Bifidobacterium animalis BB-12, Bifidobacterium animalis HN019 and Bifidobacterium animalis bI-04.

Therefore, the use of Bifidobacterium animalis CCFM1274 applied to the product containing the Ginseng Radix Et Rhizoma extract has a huge application prospect and application basis.

### BRIEF DESCRIPTION OF THE DRAWINGS

FIG. 1 shows a cell viability ratio of each group, in which different letters are marked to indicate a significant difference (P<0.05), and the same letters are marked to indicate no significant difference (P>0.05).

FIG. 2 shows an oil red concentration ratio of each group.

FIG. 3 shows an oil red staining diagram of each group, in which different letters are marked to indicate a significant difference (P<0.05), and the same letters are marked to indicate no significant difference (P>0.05).

FIG. 4 shows relative ALT and AST ratios of each group, in which different letters are marked to indicate a significant difference (P<0.05), and the same letters are marked to indicate no significant difference (P>0.05).

FIG. 5 shows a relative TG ratio of each group, in which different letters are marked to indicate a significant difference (P<0.05), and the same letters are marked to indicate no significant difference (P>0.05).

### DETAILED DESCRIPTION OF THE INVENTION

The present invention is further illustrated below in conjunction with the accompanying drawings and specific examples of the description, but the examples do not limit the present invention in any form. Unless otherwise specified, reagents, methods and apparatuses employed in the present invention are conventional reagents, methods and apparatuses in the art.

Unless otherwise specified, reagents and materials used in the following examples are commercially available.

Bifidobacterium animalis subsp.lactis CCFM1274 used in the present invention was preserved in Guangdong Microbial Collection Center (GDMCC) on Sep. 15, 2022, with a preservation number of GDMCC No. 62798, and a preservation address: 5^{th} Floor, Building 59, Yard No. 100, Xianlie Middle Road, Guangzhou.

Bifidobacterium breve CCFM1025 involved in the following examples had a preservation number of GDMCC No. 60386 and was recorded in the patent application document with a publication number of CN114533765A; Bifidobacterium breve CCFM683 had a preservation number of CGMCC No. 11828 and was recorded in the patent application document with a publication number of CN115624572A; Bifidobacterium animalis CCFM1155 had a preservation number of GDMCC No. 61495 and was recorded in the patent document with a publication number of CN113088473A; and Bifidobacterium animalis CCFM1160 had a preservation number of GDMCC No. 61500 and was recorded in the patent document with a publication number of CN113897300A.

The Ginseng Radix Et Rhizoma extract involved in the following examples was purchased from Guangzhou Infinitus; a RPMI-1640 culture solution and fetal bovine serum were purchased from gibco, Inc.; anhydrous ethanol was purchased from Sinopharm Chemical Reagent Co., Ltd.; an aspartate aminotransferase kit, an alanine aminotransferase kit and a TG kit were all purchased from Nanjing Jiancheng Company; acetic acid was analytically pure, and acetonitrile was chromatographically pure, both of which were purchased from Sinopharm Chemical Reagent Co., Ltd.; oleic acid was purchased from Sigma, USA, with a batch number of 026K13971V; and oil red 0 was purchased from Sigma, USA, with a batch number of 00625.

HepG2 cells involved in the following examples came from the cell bank of the Biotechnology Center of the School of Food Science and Technology of Jiangnan University.

The mediums involved in the following examples were as follows.

Modified MRS liquid medium (g/L): 10 g/L of peptone, 5 g/L of yeast extract, 10 g/L of beef extract, 20 g/L of glucose, 2 g/L of anhydrous sodium acetate, 2 g/L of diammonium hydrogen citrate, 2.6 g/L of K₂HPO₄·3H₂O, 0.58 g/L of MgSO₄·7H₂O, 0.25 g/L of MnSO₄·7H₂O, 1 g/L of Tween-80, 1000 g/L of distilled water, and 0.5 g/L of cysteine hydrochloride.

Modified MRS solid medium (g/L): 10 g/L of peptone, 5 g/L of yeast extract, 10 g/L of beef extract, 20 g/L of glucose, 2 g/L of anhydrous sodium acetate, 2 g/L of diammonium hydrogen citrate, 2.6 g/L of K₂HPO₄·3H₂O, 0.58 g/L of MgSO₄·7H₂O, 0.25 g/L of MnSO₄ 7H₂O, 1 g/L of Tween-80, 20 g/L of agar, 1000 g/L of distilled water, and 0.5 g/L of cysteine hydrochloride.

Fermentation base containing Ginseng Radix Et Rhizoma extract (g/L): 5 mg/mL of Ginseng Radix Et Rhizoma extract, 10 g/L of peptone, 5 g/L of yeast extract, 10 g/L of beef extract, 20 g/L of glucose, 2 g/L of anhydrous sodium acetate, 2 g/L of diammonium hydrogen citrate, 2.6 g/L of K₂IPO₄·3H₂O, 0.58 g/L of MgSO₄·7H₂O, 0.25 g/L of MnSO₄·7H₂O, 1 g/L of Tween-80, 1000 g/L of distilled water, and 0.5 g/L of cysteine hydrochloride.

The Ginseng Radix Et Rhizoma extract was an aqueous extract of Ginseng Radix Et Rhizoma, which might be purchased commercially and had a specification of 5:1 (a ratio of raw herbal materials to an extract obtained after extraction, concentration and drying was 5:1, that is, 1 g of Ginseng Radix Et Rhizoma extract was equivalent to 5 g of Ginseng Radix Et Rhizoma medicinal material).

The RPMI-1640 medium was purchased from gibco, Inc.

### Example 1: Isolation and Screening, Identification and Preservation of Strain CCFM1274

### 1. Isolation and Screening of Strain

Faeces samples from healthy adults in Inner Mongolia were diluted with sterile saline, and plate-coated and cultured on a modified MRS solid medium, and pure single colonies with consistent morphology were isolated. Different single colonies were inoculated in a modified MRS liquid medium containing a Ginseng Radix Et Rhizoma extract respectively, and a strain having a strong transformation capability for the Ginseng Radix Et Rhizoma extract was screened by analyzing the changes in the amount of rare ginsenosides in the medium after culture and fermentation, and recorded as a strain CCFM1274.

### 2. Strain Identification

The obtained strain CCFM1274 was morphologically identified with 16S rDNA molecules (sequencing was performed by Suzhou GENEWIZ Biotechnology Co., Ltd., and nucleotide sequence alignment was performed in NCBI), and the identification results indicated that the strain CCFM1274 was affiliated to Bifzdobacterium animalis subsp.lactis.

### 3. Strain Storage

Bifidobacterium animalis subsp.lactis CCFM1274 was inoculated in 5 mL of MRS liquid medium and cultured at 37° C. for 24 h; 1 mL of bacterial solution was taken and placed in a sterile centrifuge tube, and centrifuged at 8000 r/min for 3 min; an upper medium was discarded; and bacterial sludge was resuspended in 30% glycerol solution and stored at −80° C.

### 4. Strain Preservation

The Bifidobacterium animalis subsp.lactis CCFM1274 obtained was preserved in the Guangdong Microbial Collection Center (GDMCC) on Sep. 15, 2022, with a preservation number of GDMCC No. 62798, and a preservation address: 5^{th} Floor, Building 59, Yard No. 100, Xianlie Middle Road, Guangzhou.

### Example 2: Composite Fermentation Broth Obtained by Fermenting Ginseng Radix Et Rhizoma Extract with Bifidobacterium animalis Subsp.Lactis CCFM1274

### 1. Fermentation Method:

(1) the Bifidobacterium animalis subsp.lactis CCFM1274 in Example 1 was scribed on an MRS solid medium, and a flat plate was cultured inversely at 37° C. for 48 h; and a single colony was picked and inoculated into 5 mL of MRS liquid medium, and cultured at 37° C. for 48 h to prepare a seed solution.

(2) 5% (v/v) of the seed solution of Bifidobacterium animalis subsp.lactis CCFM1274 was added to a fermentation base containing a Ginseng Radix Et Rhizoma extract, and fermented at a constant temperature of 37° C. for 48 h; and after the fermentation was completed, a fermentation broth was obtained, wherein the fermentation broth had a bacterial solution concentration of 1×10⁹ CFU/mL.

(3) The fermentation broth was centrifuged at 8000 r/min for 20 min, a supernatant was collected, adjusted in pH to 7.2-7.4, and filtered and sterilized with a 0.22 m disposable filter to obtain a composite fermentation broth of the Bifidobacterium animalis subsp.lactis CCFM1274 and the Ginseng Radix Et Rhizoma extract, which was aliquoted and then stored in a −20° C. refrigerator.

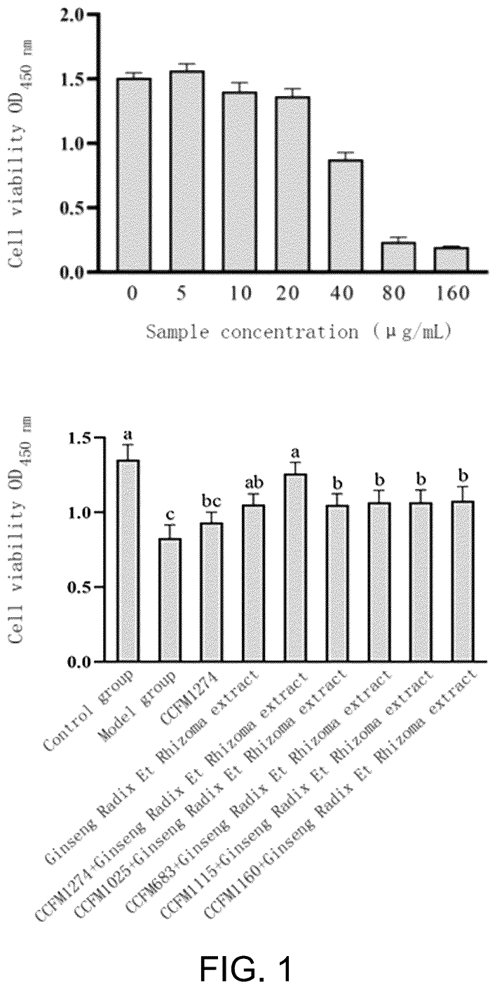

2. Ingredients of the composite fermentation broth of the Bifzdobacterium animalis subsp.lactis CCFM1274 and the Ginseng Radix Et Rhizoma extract as obtained above were analyzed, and compared with ingredients of the Ginseng Radix Et Rhizoma extract before fermentation. Results were shown in FIG. 1.

TABLE 1
Comparison of functional substances before and after fermentation
of Ginseng Radix Et Rhizoma extract with strain CCFM1274

| | | | Content increase |
|---|---|---|---|
| | Before | After | rate after |
| Detected substances | fermentation | fermentation | fermentation |
| Flavonoids (mg/ml) | 0.240 | 0.239 | — |
| Aglycone flavonoids | 0.234 | 0.264 | 12.8% |
| (mg/ml) | | | |
| Acetic acid (ml/L) | 0.987 | 3.564 | 261.1% |
| Propionic acid (ml/L) | 0.078 | — | — |
| Isobutyric acid (ml/L) | 0.152 | 0.153 | — |
| Butyric acid (ml/L) | — | 0.125 | * |
| Total content of short- | 1.217 | 3.842 | 215.7% |
| chain fatty acids | | | |

Note:
* indicated that the butyric acid was a newly added ingredient after fermentation.

From the data in the above table, it can be seen that the content of aglycone flavonoids and short-chain fatty acids (acetic acid and butyric acid) in the Ginseng Radix Et Rhizoma extract medium after fermentation with the Bifidobacterium animalis subsp.lactis CCFM1274 of the present invention significantly increased, and the aglycone flavonoids and short-chain fatty acids had an effect of improving the non-alcoholic fatty liver.

### Comparative Example

The strain in the fermentation method of Example 2 was respectively replaced with Bifidobacterium breve CCFM1025, Bifidobacterium breve CCFM683, Bifidobacterium animalis CCFM1155 and Bifidobacterium animalis CCFM1160, which were respectively prepared into a composite fermentation broth of Bifidobacterium breve CCFM1025 and the Ginseng Radix Et Rhizoma extract, a composite fermentation broth of Bifidobacterium breve CCFM683 and the Ginseng Radix Et Rhizoma extract, a composite fermentation broth of Bifidobacterium animalis CCFM1155 and the Ginseng Radix Et Rhizoma extract, and a composite fermentation broth of Bifidobacterium animalis CCFM1160 and the Ginseng Radix Et Rhizoma extract.

### Experimental Example: An Experiment of an Effect of Fermentation Broth to Alleviate Non-Alcoholic Metabolic Liver Injury

### 1. Preparation of FFA (Free Fatty Acid) Modeling Solution:

### (1) Preparation of 30% Bovine Serum Albumin (BSA) Solution

Sterile PBS buffer was preheated to 55° C., and 3.6 g of fatty acid-free bovine serum albumin powder was accurately weighed, added to a 50 mL centrifuge tube, added with 12 mL of PBS solution, and centrifuged at room temperature at 8000 r for 20 min to obtain a 30% BSA solution with a yellow and clear appearance.

### (2) Preparation of Palmitic Acid (PA) Solution

6 mL of 0.1 mol/L NaOH solution was pipetted and placed into a 50 mL centrifuge tube, added with 0.0614 g of palmitic acid granules, placed in a 75° C. water bath for 30 min, fully saponified to present a clear and transparent appearance, kept at 75° C., quickly added with 6 mL of 30% BSA solution, mixed well, and continuously dissolved at 55° C. for 30 min to obtain 20 mM palmitic acid (PA) solution with a brownish yellow and clear appearance and stable traits, which was then frozen in a −20° C. refrigerator.

### (3) Preparation of 40 mM Sodium Oleate Solution

6 mL of 0.1 mol/L NaOH solution and 500 L of oleic acid solution (1 g to 3.54 mL of ultrapure water, 1000 mmol/L) were added to a centrifuge tube, placed in a 75° C. water bath, saponified for 30 min to present a colorless and transparent appearance, added with 6 mL of 30% BSA solution under a condition of heat preservation, mixed well, and frozen in a −20° C. refrigerator.

(4) When in use, 20 mM PA solution and 40 mM sodium oleate solution (a volume ratio of 1:1) were mixed with ordinary RPMI-1640 medium (a volume ratio of 1:1) to obtain a FFA modeling solution.

### 2. Preparation of Oil Red O Staining Solution:

0.5 g of oil red O granules were dissolved in 100 mL of isopropanol, slowly heated in a water bath until completely dissolved, cooled to room temperature, filtered with filter paper, and stored at 4° C. in the dark. 2 h before use, the oil red O stock solution was mixed with distilled water at a ratio of 3:2, and slag was filtered and removed with a 0.45 m organic filter membrane.

3. Preparation of Bifidobacterium animalis Subsp.Lactis CCFM1274 Fermentation Broth:

An activated Bifidobacterium animalis subsp.lactis CCFM1274 was inoculated into a modified MRS liquid medium and cultured anaerobically at 37° C. for 24 h. The culture was inoculated into the modified MRS liquid medium at an inoculation amount of 5% (v/v), fermented anaerobically at a constant temperature of 37° C. for 48 h to obtain a bacterial solution having a concentration of 1×10⁹ CFU/mL; the bacterial solution was centrifuged at 8000 r/min for 20 min, a supernatant was collected, adjusted in pH to 7.2-7.4, and filtered and sterilized with a 0.22 μm disposable filter to obtain the Bifidobacterium animalis subsp.lactis CCFM1274 fermentation broth, which was aliquoted and then stored in a −20° C. refrigerator.

### 4. Specific Modeling Experiment

HepG2 cells were inoculated in a RPMI-1640 medium containing 10% fetal bovine serum and 1% penicillin streptomycin combination, and subcultured at a ratio of 1:3 in a 37° C., 5% CO₂ incubator. When the cell density reached 80%, different grouping processing was performed. Specific grouping data was as follows.

A blank control group was cultured with an ordinary RPMI-1640 medium for 48 h.

A model group was cultured with an ordinary RPMI-1640 medium for 24 h, and then cultured with 1.0 mmol/L FFA modeling solution for 24 h.

A Ginseng Radix Et Rhizoma extract group was cultured with an ordinary RPMI-1640 medium for 24 h, and then cultured with a Ginseng Radix Et Rhizoma extract having a concentration of 5 mg/ml and 1.0 mmol/L FFA modeling solution for 24 h.

A CCFM1274 group (i.e., a Bifidobacterium animalis subsp.lactis CCFM1274 fermentation broth group) was cultured with an ordinary RPMI-1640 medium for 24 h, and then cultured with the prepared Bifidobacterium animalis subsp.lactis CCFM1274 fermentation broth having a bacterial solution concentration of 1×10⁹ CFU/ml and 1.0 mmol/L FFA modeling solution for 24 h.

A CCFM1274+Ginseng Radix Et Rhizoma extract group was cultured with an ordinary RPMI-1640 medium for 24 h, and then cultured under the intervene of the composite fermentation broth of the Bifidobacterium animalis subsp.lactis CCFM1274 and the Ginseng Radix Et Rhizoma extract prepared in Example 2 and 1.0 mmol/L FFA modeling solution for 24 h.

A CCFM1025+Ginseng Radix Et Rhizoma extract group was cultured with an ordinary RPMI-1640 medium for 24 h, and then cultured under the intervene of the composite fermentation broth of the Bifidobacterium breve CCFM1025 and the Ginseng Radix Et Rhizoma extract prepared in Comparative example and 1.0 mmol/L FFA modeling solution for 24 h.

A CCFM683+Ginseng Radix Et Rhizoma extract group was cultured with an ordinary RPMI-1640 medium for 24 h, and then cultured under the intervene of the composite fermentation broth of the Bifidobacterium breve CCFM683 and the Ginseng Radix Et Rhizoma extract prepared in Comparative example and 1.0 mmol/L FFA modeling solution for 24 h.

A CCFM1155+Ginseng Radix Et Rhizoma extract group was cultured with an ordinary RPMI-1640 medium for 24 h, and then cultured under the intervene of the composite fermentation broth of the Bifidobacterium animalis subsp.lactis CCFM1155 and the Ginseng Radix Et Rhizoma extract prepared in Comparative example and 1.0 mmol/L FFA modeling solution for 24 h.

A CCFM1160+Ginseng Radix Et Rhizoma extract group was cultured with an ordinary RPMI-1640 medium for 24 h, and then cultured under the intervene of the composite fermentation broth of the Bifidobacterium animalis subsp.lactis CCFM1160 and the Ginseng Radix Et Rhizoma extract prepared in Comparative example and 1.0 mmol/L FFA modeling solution for 24 h.

The specific operations were as follows:

(1) HepG2 cells with a growth density of nearly 80% were digested, inoculated in a 96-well plate at a concentration of 1×10⁴ cells/well, with 100 L of cell suspension per well, and the 96-well plate was placed in an incubator for 24 h, so that after 80% of the cells were adherent, a culture solution was pipetted out and dead cells were washed away with a sterile PBS solution; and
ingredients were added according to the blank control group, the model group, the Ginseng Radix Et Rhizoma extract group, the CCFM1274 group, the CCFM1274+Ginseng Radix Et Rhizoma extract group, the CCFM1025+Ginseng Radix Et Rhizoma extract group, the CCFM683+Ginseng Radix Et Rhizoma extract group, the CCFM1155+Ginseng Radix Et Rhizoma extract group, and the CCFM1160+Ginseng Radix Et Rhizoma extract group, with 3 parallel wells being set up in each group, and continuously cultured for 24 h, and then the cell viability was detected by a CCK8 method. That is, CCK8 and 1640 culture solutions were mixed well at a ratio of 1:10, a culture solution in the 96-well plate was pipetted carefully and washed gently with a PBS solution once, 110 L of CCK8-1640 medium mixture was added to each well in the dark, and continuously cultured in an incubator for 2 h; and an absorbance value of each well at 450 nm wavelength was measured by a microplate reader.

The cell viability was calculated according to: cell viability (%)=[(As−Ab)/(Ac−Ab)]×100%.

As, represents an absorbance value for a well in the presence of cells, a CCK solution, and an administration solution.

Ab, represents an absorbance value for a well in the presence of the medium and CCK solution and in the absence of cells.

Ac, represents an absorbance value for a well in the presence of cells and the CCK solution and in the absence of administration solution.

The results were shown in FIG. 1. The results showed that when the composite fermentation broth had a concentration of 20 g/ml, it has the least impact on the cells, so 20 g/ml was chosen as the subsequent experimental concentration.

(2) HepG2 cells were inoculated in a 6-well plate at 1×10⁵ cells/well; and when the cell density was close to 70%, the cells were inoculated with different ingredients according to the blank control group, the model group, the Ginseng Radix Et Rhizoma extract group, the CCFM1274 group, the CCFM1274+Ginseng Radix Et Rhizoma extract group, the CCFM1025+Ginseng Radix Et Rhizoma extract group, the CCFM683+Ginseng Radix Et Rhizoma extract group, the CCFM1155+Ginseng Radix Et Rhizoma extract group, and the CCFM1160+Ginseng Radix Et Rhizoma extract group to perform intervene on cells, and stained with oil red O after 24 h.

Staining step: a staining solution was preheated at 37° C. A culture solution in the 12-well plate was pipetted and washed with a PBS solution twice, and the PBS solution was then pipetted away; the cells were immobilized with 4% paraformaldehyde for 30 min and pipetted away, washed with 60% isopropanol for 3 min to facilitate oil red staining; stained with oil red O for 20 min in the dark, washed with 60% isopropanol for 10 s, washed with ultrapure water for 1 min to wash off excess staining solution, and placed inversely under an inverted microscope for observation and photographing. After observation, the accumulation of lipid droplets was quantified by dissolving the stained lipid droplets with isopropanol, and the absorbance was measured at 500 nm.

(3) HepG2 cells were inoculated on a 12-well plate at 1×10⁵ cells/well; and after 24 h of cell adherence, the cells were divided into a blank control group, a model group, a Ginseng Radix Et Rhizoma extract group, a CCFM1274 group, a CCFM1274+Ginseng Radix Et Rhizoma extract group, a CCFM1025+Ginseng Radix Et Rhizoma extract group, a CCFM683+Ginseng Radix Et Rhizoma extract group, a CCFM1155+Ginseng Radix Et Rhizoma extract group, and a CCFM1160+Ginseng Radix Et Rhizoma extract group, which were continuously cultured for 24 h; a supernatant of each well was collected; and the ALT content and the AST content of the cell supernatant were measured according to instructions of ALT and AST kits. After the cells were lysed with IP lysate, the intracellular TG content was measured according to instructions of a BCA protein concentration assay kit and a TG test kit.

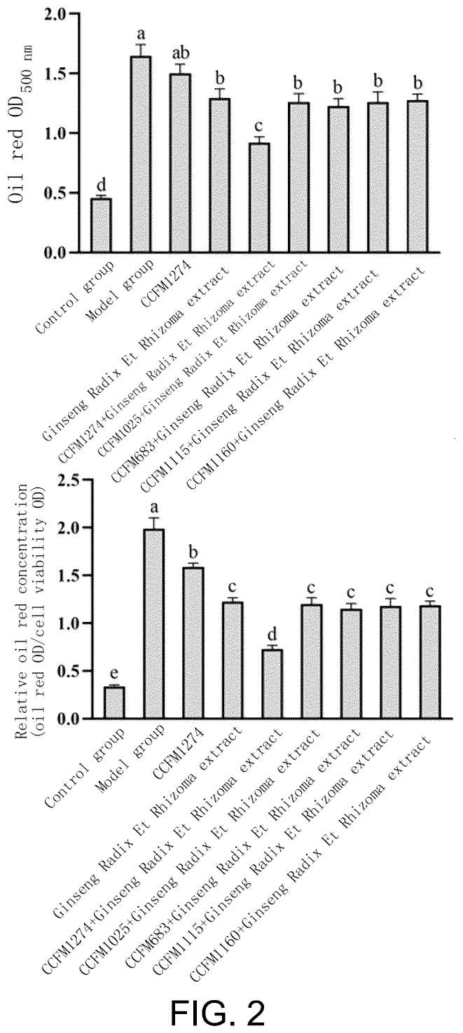

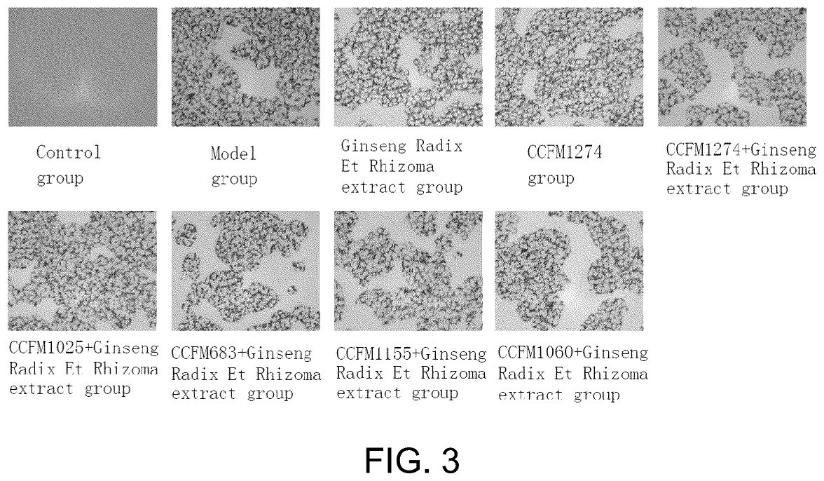

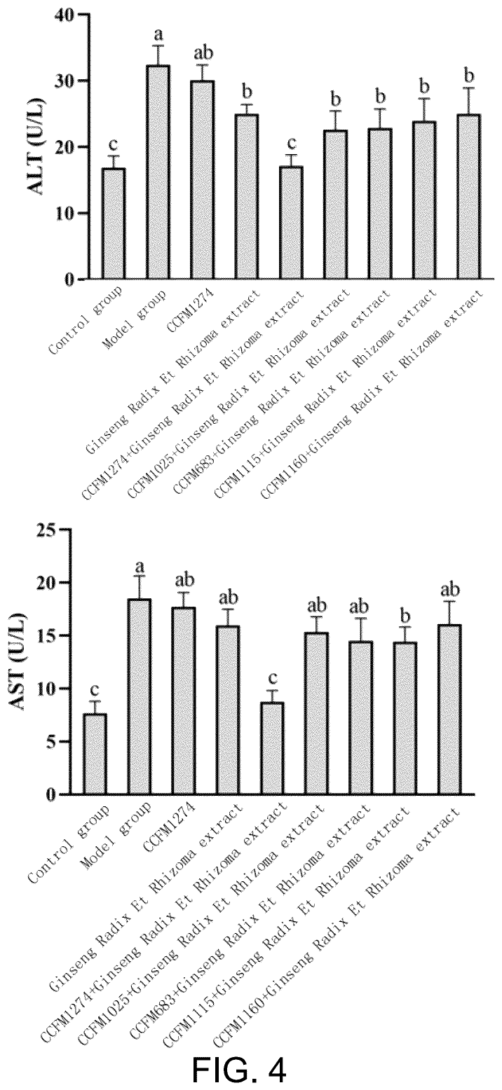

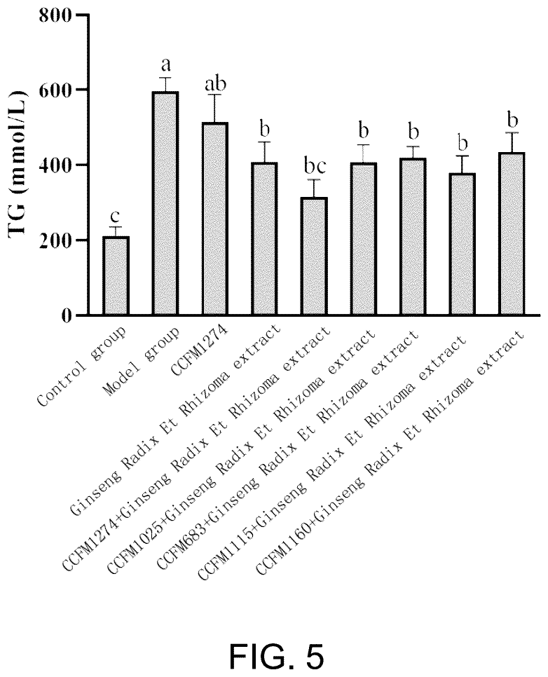

### 5. Experimental Results (Shown in FIGS. 2 to 5):

The results showed:

(1) The blank control group had an oil red absorbance of 0.45, a relative oil red concentration of 0.34, the ALT content of 16.84 U/L, the AST content of 7.66 U/L, and the TG content of 211 mmol/L.

(2) The model group had an oil red absorbance of 1.65, a relative oil red concentration of 1.99, the ALT content of 32.42 U/L, the AST content of 18.53 U/L, and the TG content of 596 mmol/L.

(3): The Bifidobacterium animalis subsp.lactis CCFM1274+Ginseng Radix Et Rhizoma extract had a staining area of oil red lower than that of other groups, and had an oil red absorbance of only 0.9 and a relative oil red concentration of only 0.73, which were also much smaller than those of other groups.

The CCFM1274+Ginseng Radix Et Rhizoma extract group had the ALT content of 17.12 U/L, the AST content of 8.75 U/L, and the TG content of 315 mmol/L.

(4) The Ginseng Radix Et Rhizoma extract group had slight reduced staining area of oil red, but had an oil red absorbance of 1.29, a relative oil red concentration of 1.22, the ALT content of 25.04 U/L, the AST content of 15.98 U/L, and the TG content of 408 mmol/L.

(5): The Bifidobacterium animalis subsp.lactis CCFM1274 group had a slightly reduced staining area of oil red, but had an oil red absorbance of 1.5, a relative oil red concentration of 1.58, the ALT content of 30.14 U/L, the AST content of 17.72 U/L, and the TG content of 514 mmol/L.

(6): The Bifidobacterium breve CCFM1025+Ginseng Radix Et Rhizoma extract had slight reduced staining area of oil red, but had an oil red absorbance of 1.26, a relative oil red concentration of 1.19, the ALT content of 22.60 U/L, the AST content of 15.34 U/L, and the TG content of 407 mmol/L.

(7): The Bifidobacterium breve CCFM683+Ginseng Radix Et Rhizoma extract had slight reduced staining area of oil red, but had an oil red absorbance of 1.22, a relative oil red concentration of 1.15, the ALT content of 22.92 U/L, the AST content of 14.54 U/L, and the TG content of 419 mmol/L.

(8): The Bifidobacterium animalis CCFM1155+Ginseng Radix Et Rhizoma extract had slight reduced staining area of oil red, but had an oil red absorbance of 1.26, a relative oil red concentration of 1.18, the ALT content of 23.98 U/L, the AST content of 14.44 U/L, and the TG content of 381 mmol/L.

(9): The Bifidobacterium animalis CCFM1160+Ginseng Radix Et Rhizoma extract had slight reduced staining area of oil red, but had an oil red absorbance of 1.28, a relative oil red concentration of 1.19, the ALT content of 25.05 U/L, the AST content of 16.12 U/L, and the TG content of 435 mmol/L.

The comparative analysis of the above results showed that the Bifidobacterium animalis subsp.lactis CCFM1274+Ginseng Radix Et Rhizoma extract group could significantly increase the proliferation activity of HepG2 cells, reduce the number of red lipid droplets in cells, increase the content of aspartate aminotransferase and alanine aminotransferase, improve a TG clearance rate, and inhibit lipid accumulation in liver cells, and had effects significantly superior to those of the single strain CCFM1274 group, the single Ginseng Radix Et Rhizoma extract group, the Bifidobacterium breve CCFM1025+Ginseng Radix Et Rhizoma extract group, the Bifidobacterium breve CCFM683+Ginseng Radix Et Rhizoma extract group, the Bifidobacterium animalis CCFM1155+Ginseng Radix Et Rhizoma extract group, and the Bifidobacterium animalis CCFM1160+Ginseng Radix Et Rhizoma extract group.

In summary, the composite fermentation broth of the Bifidobacterium animalis subsp.lactis CCFM1274 and the Ginseng Radix Et Rhizoma extract of the present invention can improve the proliferation activity of HepG2 cells, reduce the number of red lipid droplets in cells, reduce the content of aspartate aminotransferase and alanine aminotransferase, improve a TG clearance rate, and further inhibit the lipid accumulation in liver cells, so as to have the effects and application value of improving and delaying the fatty liver.

The above-mentioned examples are preferred embodiments of the present invention, but the embodiments of the present invention are not limited by the above-mentioned examples. Any other changes, modifications, substitutions, combinations, and simplifications that do not deviate from the spirit and principle of the present invention should be equivalent substitutions and are included within the protection scope of the present invention.

What is claimed is:

1. A fermented Ginseng Radix Et Rhizoma extract, obtained by subjecting a Ginseng Radix Et Rhizoma extract to fermentation with Bifidobacterium animalis subsp.lactis CCFM1274, wherein the Bifidobacterium animalis subsp.lactis CCFM1274 is preserved in Guangdong Microbial Collection Center on Sep. 15, 2022, with a preservation number of GDMCC No. 62798.

2. The fermented Ginseng Radix Et Rhizoma extract according to claim 1, wherein the fermented Ginseng Radix Et Rhizoma extract is capable of promoting an efficacy of alleviating a non-alcoholic fatty liver disease.

3. The fermented Ginseng Radix Et Rhizoma extract according to claim 1, wherein the fermented Ginseng Radix Et Rhizoma extract is capable of increasing a content of aglycone flavonoids and/or short-chain fatty acids.

4. The fermented Ginseng Radix Et Rhizoma extract according to claim 1, comprising at least one of the following functions:
(1) improving a proliferation activity of liver cells;
(2) reducing a number of red lipid droplets in the liver cells;
(3) increasing contents of aspartate aminotransferase and alanine aminotransferase in the liver cells;
(4) increasing a TG clearance rate; and
(5) inhibiting lipid accumulation in the liver cells.

5. A product for alleviating non-alcoholic fatty liver disease, comprising the fermented Ginseng Radix Et Rhizoma extract according to claim 1.

6. The product according to claim 5, wherein the product is food, drugs or health products.

7. The product according to claim 5, comprising at least one of the following functions:
(1) improving a proliferation activity of liver cells;
(2) reducing a number of red lipid droplets in the liver cells;
(3) increasing contents of aspartate aminotransferase and alanine aminotransferase in the liver cells;
(4) increasing a TG clearance rate; and
(5) inhibiting lipid accumulation in the liver cells.

8. A method for promoting increase of content of aglycone flavonoids and/or short-chain fatty acids in Ginseng Radix Et Rhizoma extract, comprising adding the Bifidobacterium animalis subsp.lactis CCFM1274 according to claim 1 to a system containing the Ginseng Radix Et Rhizoma extract for fermentation, wherein the Ginseng Radix Et Rhizoma extract fermented has a higher content of aglycone flavonoids and/or short-chain fatty acids than that in the Ginseng Radix Et Rhizoma extract before fermentation.

9. A method for promoting efficacy of Ginseng Radix Et Rhizoma extract in alleviating non-alcoholic fatty liver disease, comprising adding the Bifidobacterium animalis subsp.lactis CCFM1274 according to claim 1 to a system containing the Ginseng Radix Et Rhizoma extract for fermentation, wherein the Ginseng Radix Et Rhizoma extract fermented has better efficacy in alleviating the non-alcoholic fatty liver disease than that of the Ginseng Radix Et Rhizoma extract before fermentation.
